(19) United States
(12) Patent Application Publication
(10) Pub. No.: US 2017/0226594 A1
(43) Pub. Date: Aug. 10, 2017

(54) SHORT TANDEM REPEAT (STR) DNA FINGERPRINT METHOD AND KIT

(71) Applicant: Wafa Ali Rashed Altayari, Abu Dhabi (AE)

(72) Inventor: Wafa Ali Rashed Altayari, Abu Dhabi (AE)

(21) Appl. No.: 15/426,921

(22) Filed: Feb. 7, 2017

(30) Foreign Application Priority Data
Feb. 8, 2016 (AE) 2016/P-153

(51) Int. Cl. C12Q 1/68

(52) U.S. Cl. CPC C12Q 1/6888

(57) ABSTRACT

The present disclosure provides methods and kits for DNA fingerprinting or profiling by the combined analysis of short tandem repeats (STRs) from one or more autosomal chromosome STR loci; one or more X-chromosome STR loci and one or more Y-chromosome loci.

### CROSS-REFERENCE TO RELATED APPLICATION

This application claims priority to United Arab Emirates Patent Application No. 2016/P-153 (0000160008331236) filed 8 Feb. 2016, the entire contents of which are hereby incorporated in their entirety by reference.

### FIELD OF THE DISCLOSURE

The present disclosure relates, in some embodiments, to methods and kits for DNA fingerprinting or profiling by the combined analysis of short tandem repeats (STRs) from one or more autosomal chromosome STR loci; one or more X-chromosome STR loci and one or more Y-chromosome STR loci.

### BACKGROUND

DNA fingerprinting (also called DNA profiling or typing) can be used to identify a contributor of a genetic sample. In the field of forensics, it can be used to identify a person or persons at a crime scene or place a person at a crime scene.

DNA fingerprinting analyzes the differences in one or more regions of genomic DNA which are highly variable (i.e., polymorphic) in a population. Short tandem repeats (“STR”) are highly polymorphic regions of genomic DNA that have short repeated sequences. In STR DNA fingerprinting, one or more STR loci (i.e., regions) are targeted with sequence specific primers and amplified. The size and/or sequence of the amplified products are then analyzed. Differences in the length (i.e., number of repeats) and/or sequence of STRs may be used to discriminate between one DNA profile and another. Increasing the number of STR regions (i.e., different loci) tested increases the discrimination between one DNA profile and another. For example, the likelihood that any two individuals (except identical twins) will have the same 13-loci DNA profile can be as low as 1 in 1 billion or less.

STR analysis kits are designed to screen STR loci from either (1) autosomal chromosomes only; (2) STR loci from the Y-chromosome only; (3) STR loci from the X-chromosome only; or (4) STR loci from autosomal chromosomes and 2 or 3 STR loci from the Y-chromosome. Accordingly, multiple tests are required to screen STR loci on autosomal, X and Y chromosomes.

### SUMMARY OF THE DISCLOSURE

The present disclosure relates, according to some embodiments, to Short tandem repeat (STR) Deoxyribonucleic acid (DNA) fingerprinting methods and kits. A method of producing a short tandem repeat DNA fingerprint of a subject, the method may comprise: (a) performing at least one polymerase chain reaction (e.g., at least one polymerase chain reaction cycle) on a sample of the subject's DNA to generate an amplified sample of DNA comprising one or more amplified autosomal chromosome STR loci, one or more amplified X-chromosome STR loci, and one or more amplified Y-chromosome STR loci; and (b) fractionating the amplified sample of DNA to produce the short tandem repeat DNA fingerprint. A method may comprise comparing a short tandem repeat DNA fingerprint to a DNA fingerprint database, the database comprising at least one comparative short tandem repeat DNA fingerprint. One or more autosomal chromosome STR loci may be selected from the group consisting of D3S1358, vWA, D16S539, CSF1PO, TPDX, Amelogenin, D8S1179, D21S11, D18S51, D2S441, D19S433, THO1, FGA, D22S1045, D5S818, D135317, D7S820, SE33, D1051248, D1S1656, D125391, D2S1338, Penta D and Penta E. One or more Y-chromosome STR loci are selected from the group consisting of DYS576, DYS389I, DYS448, DYS389II, DYS19, DYS391, DYS481, DYS549, DYS533, DYS438, DYS437, DYS570, DYS635, DYS390, DYS439, DYS392, DYS643, DYS393, DYS458, DYS385a/b, DYS456 and Y-GATA-H4. One or more X-chromosome STR loci are selected from the group consisting of DXS7132, DXS7423, DXS8378, DXS10074, DXS10079, DXS10101, DXS10103, DXS10134, DX510135, DXS10146, DXS10148 and HPRTB. According to some embodiments, performing the at least one polymerase chain reaction on sample of the subject's DNA to generate an amplified sample of DNA may further comprise performing each of the at least one polymerase chain reactions in a single reaction vessel. At least one polymerase chain reaction may comprise a multiplexed Polymerase Chain Reaction. Fractionating may comprise size fractionating, wherein the size fractionating is selected from the group consisting of gel electrophoresis and capillary electrophoresis. In some embodiments, a method may comprise sequencing the amplified sample of DNA to produce a sequenced DNA sample; and comparing the sequenced DNA sample to at least one DNA sample database. In some embodiments, a method of producing a short tandem repeat DNA fingerprint of a subject, the method may comprise: (a) generating an amplified sample of DNA (e.g., by performing at least one polymerase chain reaction on a sample of the subject's DNA) comprising one or more amplified autosomal chromosome STR loci, one or more amplified X-chromosome STR loci, and one or more amplified Y-chromosome STR loci; and (b) fractionating the amplified sample of DNA to produce the short tandem repeat DNA fingerprint.

In some embodiments, a polymerase chain reaction may amplify two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty one or more, twenty two or more, or twenty three or more autosomal STRs; two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty one or more Y STRs; and two or more, three or more, four or more, five or more, six or more, seven or more, eight or more, nine or more, ten or more, eleven or more X STRs.

The present disclosure relates, in some embodiments, to a set of primers which amplify D3S1358, vWA, D16S539, CSF1PO, TPDX, Amelogenin, D8S1179, D21S11, D18S51, D2S441, D19S433, THO1, FGA, D22S1045, D5S818, D13S317, D7S820, SE33, D10S1248, D1S1656, D12S391, D2S1338, Penta D, Penta E, DXS7132, DXS7423, DXS8378, DXS10074, DXS10079, DXS10101, DXS10103, DXS10134, DXS10135, DXS10146, DXS10148, HPRTB, DYS576, DYS389I, DYS448, DYS389II, DYS19, DYS391, DYS481, DYS549, DYS533, DYS438, DYS437, DYS570, DYS635, DYS390, DYS439, DYS392, DYS643, DYS393, DYS458, DYS385a/b, DYS456 and Y-GATA-H4.

The present disclosure relates, in some embodiments, to a kit for DNA fingerprinting or profiling comprises a plurality of primers which amplify one or more autosomal chromosome STR loci selected from the group consisting D3S1358, vWA, D16S539, CSF1PO, TPDX, Amelogenin, D8S1179, D21S11, D18S51, D2S441, D19S433, THO1, FGA, D22S1045, D5S818, D13S317, D7S820, SE33, D10S1248, D1S1656, D12S391, D2S1338, PentaD and Penta E; one or more X-chromosome STR loci selected from the group consisting of DXS7132, DXS7423, DXS8378, DXS10074, DXS10079, DXS10101, DXS10103, DXS10134, DXS10135, DXS10146, DXS10148 and HPRTB and one or more Y-chromosome loci selected from the group consisting of DYS576, DYS389I, DYS448, DYS389II, DYS19, DYS391, DYS481, DYS549, DYS533, DYS438, DYS437, DYS570, DYS635, DYS390, DYS439, DYS392, DYS643, DYS393, DYS458, DYS385a/b, DYS456 and Y-GATA-H4.

The present disclosure relates, in some embodiments, to a kit for DNA fingerprinting or profiling comprising a plurality of primers which amplify the autosomal chromosome STR loci: D3S1358, vWA, D16S539, CSF1PO, TPDX, Amelogenin, D8S1179, D21S11, D18S51, D2S441, D19S433, THO1, FGA, D22S1045, D5S818, D13S317, D7S820, SE33, D10S1248, D1S1656, D12S391, D2S1338, Penta D and Penta E; X-chromosome STR loci: DXS7132, DXS7423, DXS8378, DXS10074, DXS10079, DXS10101, DXS10103, DXS10134, DXS10135, DXS10146, DXS10148 and HPRTB and Y-chromosome STR loci: DYS576, DYS389I, DYS448, DYS389II, DYS19, DYS391, DYS481, DYS549, DYS533, DYS438, DYS437, DYS570, DYS635, DYS390, DYS439, DYS392, DYS643, DYS393, DYS458, DYS385a/b, DYS456 and Y-GATA-H4. Each primer may be included in a separate formulation or may be included together in a single formulation.

A kit for DNA fingerprinting or profiling may comprise: a plurality of primers which may amplify one or more X-chromosome short tandem repeat loci; a plurality of primers which may amplify one or more Y-chromosome short tandem repeat loci; a plurality of primers which may amplify one or more autosomal chromosome short tandem repeat loci; at least one DNA polymerase; at least one DNA ladder, at least one DNA control; and at least one allelic ladder. A plurality of primers which amplify one or more Y-chromosome short tandem repeat loci may comprise DXS7132, DXS7423, DXS8378, DXS10074, DXS10079, DXS10101, DXS10103, DXS10134, DXS10135, DXS10146, DXS10148 and HPRTB, wherein a plurality of primers which amplify one or more X-chromosome short tandem repeat loci may comprise DYS576, DYS389I, DYS448, DYS389II, DYS19, DYS391, DYS481, DYS549, DYS533, DYS438, DYS437, DYS570, DYS635, DYS390, DYS439, DYS392, DYS643, DYS393, DYS458, DYS385a/b, DYS456 and Y-GATA-H4, and wherein a plurality of primers which amplify one or more autosomal chromosome short tandem repeat loci may comprise D3S1358, vWA, D16S539, CSF1PO, TPDX, Amelogenin, D8S1179, D21S11, D18S51, D2S441, D19S433, THO1, FGA, D22S1045, D5S818, D13S317, D7S820, SE33, D10S1248, D1S1656, D12S391, D2S1338, Penta D and Penta E. According to some embodiments, a DNA polymerase may comprise a taq polymerase (e.g., AmliTaq Gold DNA polymerase (Applied biosystem), DNA polymerase-gel form (Biotools), Taq polymerase recombinant (Invitrogen), Taq platinum polymerase (Invitrogen), (JumpStart Taq polymerase (Segma), Taq DNA polymerase (New England BioLabs) and Taq DNA polymerase (Promega)), a DNA ladder may comprise a 500 bp ladder or a 600 bp ladder (e.g., Internal Lane Standard 600 (ILS 600) and CC5 ILS 500 from promega, DNA Size Standard 550 (BTO) from Qiagen, GeneScan™ 500 LIZ® and GeneScan™ 600 LIZ® Size Standard from applied biosystem), a DNA control may comprise human female control DNA 9947A or male control DNA 007, and an allelic ladder may comprise allelic ladder X STRs, allelic ladder Y STRs, and allelic ladder autosomal STRs. One or more allelic ladders comprise allelic ladder X STRs, allelic ladder Y STRs, and allelic ladder autosomal STRs. One or more allelic ladders may comprise allelic ladder X STRs, allelic ladder Y STRs, and allelic ladder autosomal STRs.

### BRIEF DESCRIPTION OF THE DRAWING

Some embodiments of the disclosure may be understood by referring, in part, to the present disclosure and the accompanying drawing, wherein:

FIG. 1 illustrates a test kit according to a specific example embodiment of the disclosure.

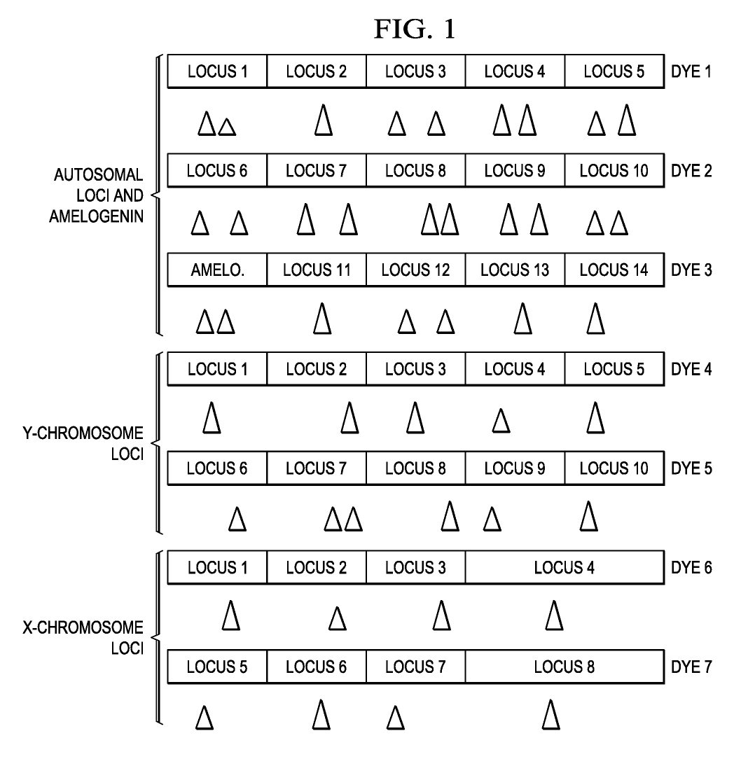

### DETAILED DESCRIPTION

The present disclosure relates, in some embodiments, to methods and kits of deoxyribonucleic acid (DNA) profiling by analysis of short tandem repeats (STRs). For example, the present disclosure provides DNA profiling methods and kits for analysis of STRs from one or more autosomal chromosome STR loci; one or more X-chromosome STR loci and one or more Y-chromosome loci. In some embodiments, the disclosure provides DNA profiling methods and kits for combined analysis of STRs from one or more autosomal chromosome STR loci; one or more X-chromosome STR loci and one or more Y-chromosome loci. A worker skilled in the art, having the benefit of the present disclosure, would readily appreciate that a combined test to screen STR loci on autosomal, X and Y chromosomes may reduce the time and effort necessary to perform the analysis. Further, the inclusion of testing additional STR loci in one test may increase the overall accuracy of the test because there will be additional data points to compare and draw conclusions from. In addition, given that forensic samples (including but not limited to samples from criminal investigations and samples from disasters including natural and man-made disasters) may be limited, a single assay to screen STR loci on autosomal, X and

Y chromosomes may be advantageous. Accordingly, such methods may be used in paternity/relationship screens and/or forensic investigations. In some embodiments, disclosed methods may provide unique solutions to challenges surrounding designing and optimizing primers and overlapping markers that have the same sizes. Analysis of three types of markers may be performed, according to some embodiments, on a spectrum instrument by Promega and/or a next generation sequencing instrument.

### STR Loci

### Autosomal STR Loci

Autosomal STR loci available for use in disclosed methods, according to some embodiments, include but are not limited to D3S1358, vWA, D16S539, CSF1PO, TPDX, Amelogenin, D8S1179, D21S11, D18S51, D2S441, D19S433, THO1, FGA, D22S1045, D5S818, D13S317, D7S820, SE33, D10S1248, D1S1656, D12S391, D2S1338, PentaD and Penta E (see for example Table 1 which details the chromosomal location, repeat motif, allelic range and polymerase chain reaction (PCR) product size of exemplary autosomal STRs).

A worker skilled in the art having the benefit of the present disclosure would readily appreciate that different databases utilize different sets of STR loci for DNA profiling. For example, the core autosomal CODIS (Combined DNA Index System) STR loci are: CSF1PO, FGA, THO1, TPDX, VWA, D3S1358, D5S818, D7S820, D8S1179, D13S317, D16S539, D18551, D21S11. The following additional 7 autosomal STRs: D1S1656, D2S441, D2S1338, D1051248, D125391, D195433, D22S1045 may be included in CODIS screens. European Standard Set (ESS) autosomal STR loci are: FGA, THO1, VWA, D1S1656, D2S441, D3S1358, D8S1179, D1051248, D125391, D18551, D21S11, D22S1045. Additional European autosomal loci include: D2S1338, D165539, D195433, SE33. The UK Core autosomal Loci are FGA, THO1, VWA, D2S1338, D3S1358, D8S1179, D16S539, D18S51, D19S433, D21S11. The German Core Autosomal Loci are: FGA, THO1, SE33, VWA, D3S1358, D8S1179, D18S51, D21S11. The Interpol Standard Set of Loci are: FGA, THO1, VWA, D3S1358, D8S1179, D18551, D21S11. Other autosomal STR loci, such as PENTA E and PENTA D are available and are used in commercial STR kits.

One or more autosomal STRs known in the art may be used in the methods of the disclosure. In some embodiments, one or more autosomal STRs selected from the group consisting of D3S1358, vWA, D165539, CSF1PO, TPDX, Amelogenin, D8S1179, D21S11, D18551, D2S441, D195433, THO1, FGA, D22S1045, D5S818, D135317, D7S820, SE33, D1051248, D1S1656, D125391, D2S1338, Penta D and Penta E are screened.

In some embodiments, two or more; three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty one or more, twenty two or more, or twenty three or more autosomal STRs are screened. In some embodiments, the autosomal STRs are selected from the group consisting of D3S1358, vWA, D165539, CSF1PO, TPDX, Amelogenin, D8S1179, D21S11, D18551, D2S441, D195433, THO1, FGA, D22S1045, D5S818, D135317, D7S820, SE33, D1051248, D1S1656, D125391, D2S1338, Penta D and Penta E. Accuracy of results may improve with each added marker, according to some embodiments. This improvement may be realized by amplifying multiple loci (e.g., autosomal, X, and Y) in a single assay in accordance with embodiments of methods and equipment disclosed herein.

In some embodiments, the following autosomal STRs are screened: CSF1PO, FGA, THO1, TPDX, VWA, D3S1358, D5S818, D7S820, D8S1179, D135317, D165539, D18551 and D21S11.

In some embodiments, the following autosomal STRs are screened: CSF1PO, FGA, THO1, TPDX, VWA, D3S1358, D5S818, D7S820, D8S1179, D13S317, D16S539, D18S51, D21S11, D1S1656, D2S441, D2S1338, D1051248, D12S391, D19S433 and D22S1045.

In some embodiments, the following autosomal STRs are screened: FGA, THO1, VWA, D1S1656, D2S441, D3S1358, D8S1179, D10S1248, D125391, D18551, D21S11 and D22S1045.

In some embodiments, the following autosomal STRs are screened: FGA, THO1, VWA, D1S1656, D2S441, D3S1358, D8S1179, D10S1248, D12S391, D18S51, D21S11, D22S1045, D2S1338, D16S539, D19S433 and SE33.

In some embodiments, the following autosomal STRs are screened: FGA, THO1, VWA, D2S1338, D3S1358, D8S1179, D16S539, D18S51, D19S433 and D21S11.

In some embodiments, the following autosomal STRs are screened: FGA, THO1, SE33, VWA, D3S1358, D8S1179, D18S51 and D21S11.

In some embodiments, the following autosomal STRs are screened: FGA, THO1, VWA, D3S1358, D8S1179, D18S51 and D21S11.

In some embodiments, the following autosomal STRs: D3S1358, vWA, D16S539, CSF1PO, TPDX, Amelogenin, D8S1179, D21S11, D18S51, D2S441, D19S433, THO1, FGA, D22S1045, D5S818, D135317, D7S820, SE33, D1051248, D1S1656, D125391, D2S1338, Penta D and Penta E are screened.

TABLE 1
Chromosomal Location, Repeat motif, Allelic Range and PCR product size of
exemplary Autosomal STRs

| | | | | PCR product sizes in |
|---|---|---|---|---|
| STR Loci | Chromosomal | | | GlobalFiler ™ kit |
| Autosomal | location | Repeat motif | Allele Range | (dye label) |
| D3S1358 | 3p21.31 | [TCTG] | 9-20 | 90.5-146.5 bp |
| | | [TCTA] | | 6-FAM |
| vWA | 12p13.31 | [TCTG] | 11-24 | 151-215 bp |
| | | [TCTA] | | 6-FAM |
| D16S539 | 16q24.1 | GATA | 5-15 | 221.5-273.5 bp |
| | | | | 6-FAM |
| CSF1PO | 5q33.3-34 | TAGA | 6-15 | 277-325 bp |
| | | | | 6-FAM |
| TPOX | 2p23-2per | GAAT | 5-15 | 332.5-384.5 bp |
| | | | | 6-FAM |
| Amelogenin | X: p22.1-22.3 Y: | NOT | NOT | VIC |
| | p11. | APPLICABLE | APPLICABLE | |
| D8S1179 | 8q24.13 | [TCTA] | 5-19 | 108.5-176.5 bp |
| | | [TCTG] | | VIC |
| D21S11 | 21q11.2-q21 | [TCTA] | 24-38 | 179.5-246.5 bp |
| | | [TCTG] | | VIC |
| D18S51 | 18q21.33 | AGAA | 7-27 | 255.5-347.5 bp |
| | | | | VIC |
| D2S441 | 2p14 | [TCTA] | 8-17 | 75-113.5 bp |
| | | [TCAA] | | NED |
| D19S433 | 19q12 | AAGG | 6-19.2 | 115.5-173.5 bp |
| | | | | NED |
| THO1 | 11p15.5 | TCAT | 4-13.3 | 174-219.5 bp |
| | | | | NED |
| FGA | 4q28 | CTTT | 13-51.2 | 221-380 bp |
| | | | | NED |
| D22S1045 | 22q12.3 | ATT | 8-19 | 83.5-126.5 bp |
| | | | | TAZ |
| D5S818 | 5q21-31 | AGAT | 7-18 | 133.5-189.5 bp |
| | | | | TAZ |
| D13S317 | 13q22-31 | TATC | 5-16 | 197-249 bp |
| | | | | TAZ |
| D7S820 | 7q11.21-22 | GATA | 6-15 | 256.5-304.5 bp |
| | | | | TAZ |
| SE33 | 6q14 | | 4.2-37 | TAZ |
| D10S1248 | 10q26.3 | GGAA | 8-19 | 80-132 bp |
| | | | | SID |
| D1S1656 | 1q42.2 | TAGA | 9-20.3 | 154-209.5 bp |
| | | | | SID |
| D12S391 | 12p13.2 | [AGAT] | 14-27 | 211-270.5 bp |
| | | [ACAC] | | SID |
| D2S1338 | 2q35 | [TGCC] | 11-28 | 275.5-355.5 bp |
| | | [TTCC] | | SID |
| Penta D | 21q | AAAGA | 2.2-17 | 376-449 |
| | | | | JOE |
| Penta E | 15q | [AAAGA] | 5-24 | 379-474 |
| | | | | fluorescein |

### Y STR Loci

Y STR loci available for use in disclosed methods, according to some embodiments, include but are not limited to DYS576, DYS389I, DYS448, DYS389II, DYS19, DYS391, DYS481, DYS549, DYS533, DYS438, DYS437, DYS570, DYS635, DYS390, DYS439, DYS392, DYS643, DYS393, DYS458, DYS385a/b, DYS456 and Y-GATA-H4. See for example Table 2 which details the chromosomal location, repeat motif, allelic range and PCR product size of exemplary Y STRs.

One or more Y STRs known in the art may be used in the methods of the disclosure. In some embodiments, one or more Y STR loci selected from the group consisting of DYS576, DYS389I, DYS448, DYS389II, DYS19, DYS391, DYS481, DYS549, DYS533, DYS438, DYS437, DYS570, DYS635, DYS390, DYS439, DYS392, DYS643, DYS393, DYS458, DYS385a/b, DYS456 and Y-GATA-H4 are screened.

In some embodiments, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty one or more, Y STRs are screened. In some embodiments, the Y STRs are selected from the group consisting of DYS576, DYS389I, DYS448, DYS389II, DYS19, DYS391, DYS481, DYS549, DYS533, DYS438, DYS437, DYS570, DYS635, DYS390, DYS439, DYS392, DYS643, DYS393, DYS458, DYS385a/b, DYS456 and Y-GATA-H4. Accuracy of results may improve with each added marker, according to some embodiments. This improvement may be realized by amplifying multiple loci (e.g., autosomal, X, and Y) in a single assay in accordance with embodiments of methods and equipment disclosed herein.

In some embodiments, the following Y STRs are screened DYS576, DYS389I, DYS448, DYS389II, DYS19, DYS391, DYS481, DYS549, DYS533, DYS438, DYS437, DYS570, DYS635, DYS390, DYS439, DYS392, DYS643, DYS393, DYS458, DYS385a/b, DYS456 and Y-GATA-H4.

TABLE 2
Chromosomal Location, Repeat motif, Allelic Range
and PCR product size of exemplary Y STRs

| | | | | PCR product sizes |
|---|---|---|---|---|
| | Chromosomal | Repeat | Allele | in PowerPlex ® |
| STR Loci Y | location | motif | Range | 23Y kit (dye label) |
| DYS576 | Yp11.2 | AAAG | 11-23 | 97-145 |
| | | | | Fluorescein |
| DYS389I | Yq11.221 | (TCTG) | 9-17 | 147-179 |
| | | (TCTA) | | Fluorescein |
| DYS448 | Yq11.223 | AGAGAT | 14-24 | 196-256 |
| | | | | Fluorescein |
| DYS389II | Yq11.221 | (TCTG) | 24-35 | 259-303 |
| | | (TCTA) | | Fluorescein |
| DYS19 | YP11.2 | TAGA | 9-19 | 312-352 |
| | | | | Fluorescein |
| DYS391 | Yq11.21 | TCTA | 5-16 | 86-130 |
| | | | | JOE |
| DYS481 | Yp11.2 | CTT | 17-32 | 139-184 |
| | | | | JOE |
| DYS549 | Yp11.2 | GATA | 7-17 | 198-238 |
| | | | | JOE |
| DYS533 | Yq11.221 | ATCT | 7-17 | 245-285 |
| | | | | JOE |
| DYS438 | Yq11.221 | TTTTC | 6-16 | 293-343 |
| | | | | JOE |
| DYS437 | Yq11.221 | TCTA | 11-18 | 344-380 |
| | | | | JOE |
| DYS570 | Yp11.2 | TTTC | 10-25 | 90-150 |
| | | | | TMR-ET |
| DYS635 | Yq11.221 | TSTA | 15-28 | 150-202 |
| | | compound | | TMR-ET |
| DYS390 | Yq11.221 | (TCTA) | 17-29 | 207-255 |
| | | (TCTG) | | TMR-ET |
| DYS439 | Yq11.221 | AGAT | 6-17 | 263-307 |
| | | | | TMR-ET |
| DYS392 | Yq11.223 | TAT | 4-20 | 314-362 |
| | | | | TMR-ET |
| DYS643 | Yq11.221 | CTTTT | 6-17 | 368-423 |
| | | | | TMR-ET |
| DYS393 | Yq12 | AGAT | 7-18 | 101-145 |
| | | | | CXR-ET |
| DYS458 | Yp11.2 | GAAA | 10-24 | 159-215 |
| | | | | CXR-ET |
| DYS385a/b | Yq11.222 | GAAA | 7-28 | 223-307 |
| | | | | CXR-ET |
| DYS456 | Yp11.2 | AGAT | 11-23 | 316-364 |
| | | | | CXR-ET |
| Y-GATA- | Yq11.221 | TAGA | 8-18 | 374-414 |
| H4 | | | | CXR-ET |

### X STR Loci

X STR loci available for use in disclosed methods, according to some embodiments, include but are not limited to DXS7132, DXS7423, DXS8378, DXS10074, DXS10079, DXS10101, DXS10103, DXS10134, DXS10135, DXS10146, DXS10148 and HPRTB. See for example Table 3 which details the chromosomal location, repeat motif, allelic range and PCR product size of exemplary X STRs.

In some embodiments, one or more X STR loci are screened. In some embodiments, the one or more X STRs are selected from the group consisting of DXS7132, DXS7423, DXS8378, DXS10074, DXS10079, DXS10101, DXS10103, DXS10134, DXS10135, DXS10146, DXS10148 and HPRTB.

In some embodiments, two or more, three or more, four or more, five or more, six or more, seven or more, eight or more, nine or more, ten or more, eleven or more X STRs are screened. In some embodiments, the X STRs are selected from the group consisting of DXS7132, DXS7423, DXS8378, DXS10074, DXS10079, DXS10101, DXS10103, DXS10134, DXS10135, DXS10146, DXS10148 and HPRTB. Accuracy of results may improve with each added marker, according to some embodiments. This improvement may be realized by amplifying multiple loci (e.g., autosomal, X, and Y) in a single assay in accordance with embodiments of methods and equipment disclosed herein.

In some embodiments, the following X STRs are screened: DXS7132, DXS7423, DXS8378, DXS10074, DXS10079, DXS10101, DXS10103, DXS10134, DXS10135, DXS10146, DXS10148 and HPRTB.

TABLE 3
Chromosomal Location, Repeat motif, Allelic Range and PCR product size of
exemplary X STRs

| | | | | Investigator |
|---|---|---|---|---|
| | | | | Argus-x-12 |
| STR Loci | Chromosomal | | Allele | kit |
| X | location | Repeat motif | Range | (dye label) |
| DXS7132 | Xq11.2 | [TCTA]₁₃ | 8-20 | 6-FAM |
| DXS7423 | Xq28 | [TCCA]3 TCTGTCCT [TCCA]12 | 8-19 | BTY |
| DXS8378 | Xp22.31 | [CTAT]12 | 7-15 | 6-FAM |
| DXS10074 | Xq12 | [AAGA]14 | 4-21 | BTG |
| DXS10079 | Xq12 | [AGAG]3 TGAAAGAG [AGAA]17 | 14-25 | BTY |
| | | AGAG [AGAA]3 | | |
| DXS10101 | Xq26.2 | [AAAG]3 GAAAGAAG [GAAA]3 A | 24-38 | BTG |
| | | [GAAA]4 AAGA [AAAG]5 | | |
| | | AAAAAGAA [AAAG]13 AA | | |
| DXS10103 | Xq26.2 | [TAGA]2 CTGA | 15-21 | 6-FAM |
| | | [CAGA][TAGA]11[CAGA]4[TAGA] | | |
| DXS10134 | Xq28 | [GAAA]3 GAGA [GAAA]4 AA | 28-46.1 | 6-FAM |
| | | [GAAA]GAGA [GAAA]4 GAGA | | |
| | | [GACAGA]3 [GAAA] GTAA | | |
| | | [GAAA]3 AAA [GAAA]4 AAA | | |
| | | [GAAA]15 | | |
| XS10135 | Xp22.31 | [AAGA]3 GAAAG [GAAA]20 | 13-39.2 | BTG |
| DXS10146 | Xq28 | [TTCC]3 T [TTCC]3 TTTC | 24-46.2 | BTY |
| | | CTCCCTTCC [TTCC][TCCC] | | |
| | | TTCTTCTTTC [TTCC]2 TTTCTT | | |
| | | [CTTT]2 CTTC [CTTT]10 T [CTTT]2 | | |
| DXS10148 | Xp22.31 | [GGAA]4[AAGA]12[AAAG]4 | | BTR |
| | | N8[AAGG]2 | | |
| HPRTB | Xq26.2 | [AGAT]12* | 6-19 | BTR |

### Methods

In some embodiments, the present disclosure provides methods for DNA fingerprinting or profiling by the combined analysis of STRs from one or more autosomal chromosome STR loci, one or more X-chromosome STR loci and one or more Y-chromosome loci. The present disclosure also relates to methods of producing a short tandem repeat DNA fingerprint of a subject. In some embodiments, a subject may be a mammal (e.g, human or non-human animal). In particular, there is provided a method of determining a DNA profile comprising: contacting a genomic DNA with a plurality of primers which amplify one or more autosomal chromosome STR loci, one or more X-chromosome STR loci and one or more Y-chromosome loci to generate amplification products; and analysing the amplification products to determine the DNA profile. In some embodiments, a method of producing a short tandem repeat DNA fingerprint of a subject may comprise: (a) performing at least one polymerase chain reaction on sample of the subject's DNA to generate an amplified sample of DNA comprising one or more amplified autosomal chromosome STR loci, one or more amplified X-chromosome STR loci, and one or more amplified Y-chromosome STR loci; and (b) fractionating the amplified sample of DNA to produce the short tandem repeat DNA fingerprint. A method of producing a short tandem repeat DNA fingerprint of a subject may comprise comparing the short tandem repeat DNA fingerprint to a DNA fingerprint database, the database comprising at least one comparative short tandem repeat DNA fingerprint. A DNA fingerprint database may comprise fingerprint(s) from one or more healthy reference subjects (e.g., a population of healthy subjects) and/or fingerprint(s) from one ore more reference subjects (e.g., a population with a diagnosed condition(s)). A DNA fingerprint database may comprise fingerprint(s) for criminology and/or genetic genealogy. In some embodiments, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty one or more, twenty two or more, or twenty three or more autosomal STRs are screened; two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty one or more Y STRs are screened; and two or more, three or more, four or more, five or more, six or more, seven or more, eight or more, nine or more, ten or more, eleven or more X STRs are screened. In some embodiments, the autosomal STR loci are selected from the loci set forth in Table 1, the Y STR loci are selected from the loci set forth in Table 2 and the X STR loci are selected from the loci set forth in Table 3.

According to some embodiments of the present disclosure, a method of determining a DNA profile comprises: contacting a genomic DNA with a plurality of primers which amplify one or more autosomal chromosome STR loci selected from the group consisting of D3S1358, vWA, D16S539, CSF1PO, TPDX, Amelogenin, D8S1179, D21S11, D18S51, D2S441, D19S433, THO1, FGA, D22S1045, D5S818, D135317, D7S820, SE33, D1051248, D1S1656, D125391, D2S1338, Penta D and Penta E; one or more X-chromosome STR loci selected from the group consisting of DXS7132, DXS7423, DXS8378, DXS10074, DXS10079, DXS10101, DXS10103, DXS10134, DXS10135, DXS10146, DXS10148 and HPRTB; and one or more Y-chromosome loci selected from the group consisting of DYS576, DYS389I, DYS448, DYS389II, DYS19, DYS391, DYS481, DYS549, DYS533, DYS438, DYS437, DYS570, DYS635, DYS390, DYS439, DYS392, DYS643, DYS393, DYS458, DYS385a/b, DYS456 and Y-GATA-H4 to generate amplification products; and analysing the amplification products to determine the DNA profile.

In some embodiments of the present disclosure, there is a method of determining a DNA profile comprising: contacting a genomic DNA with a plurality of primers which amplify autosomal chromosome STR loci: D3S1358, vWA, D165539, CSF1PO, TPDX, Amelogenin, D8S1179, D21S11, D18S51, D2S441, D19S433, TH01, FGA, D22S1045, D5S818, D13S317, D7S820, SE33, D10S1248, D1S1656, D12S391, D2S1338, Penta D and Penta E; X-chromosome STR loci: DXS7132, DXS7423, DXS8378, DXS10074, DXS10079, DXS10101, DXS10103, DXS10134, DXS10135, DXS10146, DXS10148 and HPRTB; and Y-chromosome loci: DYS576, DYS389I, DYS448, DYS389II, DYS19, DYS391, DYS481, DYS549, DYS533, DYS438, DYS437, DYS570, DYS635, DYS390, DYS439, DYS392, DYS643, DYS393, DYS458, DYS385a/b, DYS456 and Y-GATA-H4 to generate amplification products; and analysing the amplification products to determine the DNA profile.

Genomic DNA Sample:

A worker skilled in the art having the benefit of the present disclosure would readily appreciate that the genomic DNA may be isolated from a variety of samples including but not limited to hair, feces, blood, tissue, urine, saliva, cheek cells, vaginal cells, skin, bone, tooth, buccal sample, amniotic fluid containing placental cells, and amniotic fluid containing fetal cells and semen. In some embodiments, the sample may originate from a crime scene, a sample associated with a crime scene, a sample taken from a suspect, a reference sample or a sample taken from a human under consideration. In other embodiments, the sample may be an archeological sample, a sample from a disaster scene, a sample from a mass fatality, a maternity sample, a paternity sample, a missing person sample. Genomic DNA can be prepared for use in the methods of the present disclosure using any procedures for sample preparation that are compatible with the subsequent amplification of DNA. Many such procedures are known by those skilled in the art.

Amplification:

A worker skilled in the art having the benefit of the present disclosure would readily appreciate appropriate methods for the amplification of STR loci. Such methods include PCR (i.e., non-multiplexed) and multiplexed PCR. Accordingly, the primers may be in separate formulations or in a single formulation allowing for co-amplification of the STRs. A worker skilled in the art, would appreciate that in non-multiplexed PCR reactions each locus is amplified separately and the amplicons that are produced are combined prior to analysis. Such a worker would further appreciate that multiplex PCR reactions allows for the co-amplification of the STR markers. Appropriate primers for use in the amplification reaction can be determined by a worker skilled in the art through optimizations and validations. Such a worker would further appreciate that the primers may be labelled, for example with fluorescent dyes. In particular, A worker skilled in the art having the benefit of the present disclosure would readily appreciate that multiple STR loci may be co-amplified because of the use of different coloured fluorescent dye labels and the different sizes of resulting amplification products.

Accordingly, in some embodiments of the present disclosure, the methods of determining a DNA profile comprise a multiplex PCR reaction which allows for the co-amplification of the one or more autosomal chromosome STR loci; one or more X-chromosome STR loci and one or more Y-chromosome loci. In some embodiments of the present disclosure, there is a method of determining a DNA profile comprising: contacting genomic DNA with a plurality of primers which co-amplify autosomal chromosome STR loci: D3S1358, vWA, D16S539, CSF1PO, TPDX, Amelogenin, D8S1179, D21S11, D18S51, D2S441, D19S433, THO1, FGA, D22S1045, D5S818, D13S317, D7S820, SE33, D10S1248, D1S1656, D12S391, D2S1338, PentaD and Penta E; X-chromosome STR loci: DXS7132, DXS7423, DXS8378, DXS10074, DXS10079, DXS10101, DXS10103, DXS10134, DXS10135, DXS10146, DXS10148 and HPRTB; and Y-chromosome loci: DYS576, DYS389I, DYS448, DYS389II, DYS19, DYS391, DYS481, DYS549, DYS533, DYS438, DYS437, DYS570, DYS635, DYS390, DYS439, DYS392, DYS643, DYS393, DYS458, DYS385a/b, DYS456 and Y-GATA-H4 to generate amplification products; and analysing the amplification products to determine the DNA profile.

Analysis:

Techniques for the analysis of the amplification products of STR loci to determine a genetic profile are known in the art. In an exemplary method, following amplification, the size of the amplification products of the STR loci (as referred to as STR amplicons) are determined by using gel or capillary electrophoresis. In some embodiments, an amplified sample of DNA may be fractionated to produce the short tandem repeat DNA fingerprint. Fractionating may comprise any desired means of separating molecules in a mixture on the basis of physical, chemical, and/or electrical properties. For example, fractionation may include size fractionating nucleic acids. Size fractionation may include techniques such as gel electrophoresis and capillary electrophoresis. Briefly, as the primers used in the amplification are fluorescently labelled, each STR amplicon is also labelled. Accordingly, the DNA profile may be determined by separating the labelled STR amplicons with gel or capillary electrophoresis and comparing to an internal size standard (e.g., a 600 bp ladder will suffice where all loci are within the range of 100 bp to 500 bp).

According to some embodiments, an amplified sample of DNA may be sequenced to produce a sequenced DNA sample. Analysis may include sequencing of the amplicons. DNA sequencing comprises Maxam-Gilbert sequencing, Sanger sequencing, Shotgun sequencing, and Bridge PCR. In some embodiments, methods of the disclosure may further comprise comparing the profile of the genomic sample to a known profile thereby allowing for the determination of the contributor of the genomic sample. A sequenced DNA sample may be compared to at least one DNA sample database, wherein samples can be compared (e.g., matched). A DNA database may comprise a Combined DNA Index System (CODIS), a National DNA Database (NDNAD), a National Criminal Investigation DNA Database (NCIDD), a National DNA Data Bank (NDDB), a Convicted Offender index (COI), a German Federal Police (BKA), an Israel Police DNA Index System, and custom data bases.

### Kits

In some embodiments, the present disclosure provides kits for DNA fingerprinting or profiling by the combined analysis of STRs from one or more autosomal chromosome STR loci; one or more X-chromosome STR loci and one or more Y-chromosome loci. In particular, in some embodiments, the present disclosure provides kits for DNA fingerprinting or profiling utilizing the methods described herein. In some embodiments, using a single kit as disclosed conserves sample relative to running three separate analysis, one for each of autosomal STRs, X-chromosome STRs, and Y-chromosome STRs. This conservation may be desirable in cases where available sample material is limited. One of ordinary skill in the art having the benefit of the present disclosure would appreciate that simply combining the analysis of autosomal STRs, X-chromosome STRs, and Y-chromosome STRs would be problematic where each requires different PCR reaction conditions and/or overlapping markers are used. For example, kits configured to separately amplify autosomal STRs, X-chromosome STRs, and Y-chromosome markers that each result in products between 100 pb and 500 bp, if combined, would obscure, rather than clarify, identification because of the difficulty of resolving overlapping markers. The present disclosure relates, in some embodiments, to kits configured to operate under a single set of reaction conditions and refrain from using overlapping markers to concurrently (e.g., in a single reaction vessel) and reliably analyze autosomal STRs, X-chromosome STRs, and Y-chromosome STRs. Arrangements of loci selected for amplification may differ, according to some embodiments, in a kit for concurrent analysis relative to kits for separate analysis.

In some embodiments, the present disclosure provides a kit for DNA fingerprinting or profiling comprising a plurality of primers which amplify one or more autosomal chromosome STR loci; one or more X-chromosome STR loci and one or more Y-chromosome loci. In some embodiments, the autosomal STR loci are selected from the loci set forth in Table 1, the Y STR loci are selected from the loci set forth in Table 2 and the X STR loci are selected from the loci set forth in Table 3. In some embodiments the plurality of primers are formulated for a multiplex PCR co-amplification reaction.

Accordingly, in some embodiments there is provided a set of primers which amplify the following STR loci: one or more autosomal STR loci selected from the loci set forth in Table 1, one or more Y STR loci selected from the loci set forth in Table 2 and one or more X STR loci selected from the loci set forth in Table 3.

In some embodiments, the present disclosure provides a kit for DNA fingerprinting or profiling comprising a plurality of primers which amplify one or more autosomal chromosome STR loci selected from the group consisting of D3S1358, vWA, D16S539, CSF1PO, TPDX, Amelogenin, D8S1179, D21S11, D18S51, D2S441, D19S433, TH01, FGA, D22S1045, D5S818, D13S317, D7S820, SE33, D10S1248, D1S1656, D12S391, D2S1338, PentaD and Penta E; one or more X-chromosome STR loci selected from the group consisting of DXS7132, DXS7423, DXS8378, DXS10074, DXS10079, DXS10101, DXS10103, DXS10134, DXS10135, DXS10146, DXS10148 and HPRTB; and one or more Y-chromosome loci selected from the group consisting of DYS576, DYS389I, DYS448, DYS389II, DYS19, DYS391, DYS481, DYS549, DYS533, DYS438, DYS437, DYS570, DYS635, DYS390, DYS439, DYS392, DYS643, DYS393 DYS458, DYS385a/b, DYS456 and Y-GATA-H4.

In some embodiments, the present disclosure provides a kit for DNA fingerprinting or profiling comprising a plurality of primers which amplify the autosomal chromosome STR loci: D3S1358, vWA, D16S539, CSF1PO, TPDX, Amelogenin, D8S1179, D21S11, D18S51, D2S441, D19S433, THO1, FGA, D22S1045, D5S818, D13S317, D7S820, SE33, D10S1248, D1S1656, D12S391, D2S1338, Penta D and Penta E; X-chromosome STR loci: DXS7132, DXS7423, DXS8378, DXS10074, DXS10079, DXS10101, DXS10103, DXS10134, DXS10135, DXS10146, DXS10148 and HPRTB; and Y-chromosome STR loci: DYS576, DYS389I, DYS448, DYS389II, DYS19, DYS391, DYS481, DYS549, DYS533, DYS438, DYS437, DYS570, DYS635, DYS390, DYS439, DYS392, DYS643, DYS393, DYS458, DYS385a/b, DYS456 and Y-GATA-H4.

A worker skilled in the art having the benefit of the present disclosure would readily appreciated the primers may be labelled, with for example fluorescent dye. Non-limiting examples of dyes include 6-FAM, BTG, BTY, BTR, BTO, VIC, NED, TAZ, SID, JOE, TMR-ET and CXR-ET. Such a worker would readily appreciate that different coloured fluorescent dye labels may be used for one or more of the primers in the kit.

In some embodiments, a kit includes a size standard. In various embodiments, a kit includes an allelic ladder. In some embodiments, amplification reagents and/or electrophoresis reagents such as a sufficient quantity of enzyme for amplification, amplification buffer to facilitate the amplification, a divalent cation solution to facilitate enzyme activity, dNTPs for strand extension during amplification, loading solution for preparation of the amplified material for electrophoresis, genomic DNA as a template control, a size marker to insure that materials migrate as anticipated in the separation medium, and a protocol and manual to educate the user and limit error in use may be included in the kits of the disclosure in any combination or selection. The amounts of the various reagents in the kits also can be varied depending upon a number of factors, such as the optimum sensitivity of the process. In some embodiments, the amplification components are provided in a separate kit from the electrophoresis components. In alternative embodiments, the amplification components and electrophoresis components are provided in one kit. Those in the art understand that the detection techniques employed are generally not limiting. Rather, a wide variety of detection means are within the scope of the disclosed methods and kits, provided that they allow the presence or absence of an amplicon to be determined.

In some embodiments, kits comprise one or more of the following components: PCR Reaction Mix, Primer Mix X STRs, Primer Mix Y STRs, Primer Mix autosomal STRs, Multi Taq2 DNA Polymerase, Control DNA (such as 9947A (female) or 9948 (male) control DNAs validated by NIST and used by the FBI), DNA size standard, Allelic ladder X STRs, Allelic ladder Y STRs, Allelic ladder autosomal STRs, Nuclease-free water, Matrix components, Hi-Di Formamide and Matrix Standard multi cap.

In some embodiments, kits comprise the following components: PCR Reaction Mix, Primer Mix X STRs, Primer Mix Y STRs, Primer Mix autosomal STRs, Multi Taq2 DNA Polymerase, Control DNA, DNA size standard, Allelic ladder X STRs, Allelic ladder Y STRs, Allelic ladder autosomal STRs, Nuclease-free water, Matrix components, Hi-Di Formamide and Matrix Standard multi cap.

Although the disclosure has been described with reference to certain some embodiments, various modifications thereof will be apparent to those skilled in the art without departing from the spirit and scope of the disclosure. Accordingly, the manner of carrying out the disclosure as shown and described is to be construed as illustrative only. Persons skilled in the art may make various changes in the selection, number, and/or arrangement of STRs/loci without departing from the scope of the instant disclosure. Each disclosed method and method step may be performed in association with any other disclosed method or method step and in any order according to some embodiments. Where the verb “may” appears, it is intended to convey an optional and/or permissive condition, but its use is not intended to suggest any lack of operability unless otherwise indicated. Where open terms such as “having” or “comprising” are used, one of ordinary skill in the art having the benefit of the instant disclosure will appreciate that the disclosed features or steps optionally may be combined with additional features or steps. Such option may not be exercised and, indeed, in some embodiments, disclosed systems, compositions, apparatuses, and/or methods may exclude any other features or steps beyond those disclosed herein. Elements, compositions, devices, systems, methods, and method steps not recited may be included or excluded as desired or required. Persons skilled in the art may make various changes in methods of preparing and using a composition, device, and/or system of the disclosure. For example, a composition, device, and/or system may be prepared and or used as appropriate for animal and/or human use (e.g., with regard to sanitary, infectivity, safety, toxicity, biometric, and other considerations). Also, where ranges have been provided, the disclosed endpoints may be treated as exact and/or approximations as desired or demanded by the particular embodiment. Where the endpoints are approximate, the degree of flexibility may vary in proportion to the order of magnitude of the range. Accordingly, the foregoing disclosure is intended to be illustrative, but not limiting, of the scope of the disclosure as illustrated by the appended claims.

1. A method of producing a short tandem repeat DNA fingerprint of a subject, the method comprising:
(a) performing at least one polymerase chain reaction on sample of the subject's DNA to generate an amplified sample of DNA comprising one or more amplified autosomal chromosome short tandem repeat loci, one or more amplified X-chromosome short tandem repeat loci, and one or more amplified Y-chromosome short tandem repeat loci; and
(b) fractionating the amplified sample of DNA to produce the short tandem repeat DNA fingerprint.

2. The method of claim 1, further comprising comparing the short tandem repeat DNA fingerprint to a DNA fingerprint database, the database comprising at least one comparative short tandem repeat DNA fingerprint.

3. The method of claim 1, wherein the one or more autosomal chromosome short tandem repeat loci are selected from the group consisting of D3S1358, vWA, D165539, CSF1PO, TPDX, Amelogenin, D8S1179, D21S11, D18551, D2S441, D195433, THO1, FGA, D22S1045, D5S818, D135317, D7S820, SE33, D10S1248, D1S1656, D125391, D2S1338, Penta D and Penta E.

4. The method of claim 1, wherein the one or more Y-chromosome short tandem repeat loci are selected from the group consisting of DYS576, DYS389I, DYS448, DYS389II, DYS19, DYS391, DYS481, DYS549, DYS533, DYS438, DYS437, DYS570, DYS635, DYS390, DYS439, DYS392, DYS643, DYS393, DYS458, DYS385a/b, DYS456 and Y-GATA-H4.

5. The method of claim 1, wherein the one or more X-chromosome short tandem repeat loci are selected from the group consisting of DXS7132 (SEQ ID NO: 1), DXS7423 (SEQ ID NO: 2), DXS8378 (SEQ ID NO: 3), DXS10074 (SEQ ID NO: 4), DXS10079 (SEQ ID NO: 5), DXS10101 (SEQ ID NO: 6), DXS10103 (SEQ ID NO: 7), DXS10134 (SEQ ID NO: 8), DXS10135 (SEQ ID NO: 9), DXS10146 (SEQ ID NO: 10), DXS10148 (SEQ ID NO: 11), and HPRTB (SEQ ID NO: 12).

6. The method of claim 1, the at least one polymerase chain reaction comprises a multiplexed Polymerase Chain Reaction.

7. The method of claim 1, wherein the fractionating comprises size fractionating.

8. The method of claim 7, wherein the size fractionating is selected from the group consisting of gel electrophoresis and capillary electrophoresis.

9. The method of claim 1, further comprising sequencing the amplified sample of DNA to produce a sequenced DNA sample; and comparing the sequenced DNA sample to at least one DNA sample database.

10. A kit for DNA fingerprinting or profiling comprising a plurality of primers which amplify one or more autosomal chromosome short tandem repeat loci, one or more X-chromosome short tandem repeat loci, and one or more Y-chromosome loci.

11. The kit of claim 10, the one or more autosomal chromosome short tandem repeat loci are selected from the group consisting of D3S1358, vWA, D165539, CSF1PO, TPDX, Amelogenin, D8S1179, D21S11, D18551, D2S441, D195433, THO1, FGA, D22S1045, D5S818, D135317, D7S820, SE33, D10S1248, D1S1656, D125391, D2S1338, Penta D and Penta; the one or more Y-chromosome short tandem repeat loci are selected from the group consisting of DYS576, DYS389I, DYS448, DYS389II, DYS19, DYS391, DYS481, DYS549, DYS533, DYS438, DYS437, DYS570, DYS635, DYS390, DYS439, DYS392, DYS643, DYS393, DYS458, DYS385a/b, DYS456 and Y-GATA-H4; and the one or more X-chromosome short tandem repeat loci are selected from the group consisting of DXS7132 (SEQ ID NO: 1), DXS7423 (SEQ ID NO: 2), DXS8378 (SEQ ID NO: 3), DXS10074 (SEQ ID NO: 4), DXS10079 (SEQ ID NO: 5), DXS10101 (SEQ ID NO: 6), DXS10103 (SEQ ID NO: 7), DXS10134 (SEQ ID NO: 8), DXS10135 (SEQ ID NO: 9), DXS10146 (SEQ ID NO: 10), DXS10148 (SEQ ID NO: 11), and HPRTB (SEQ ID NO: 12).

12. A set of primers which amplify one or more autosomal chromosome short tandem repeat loci selected from the group consisting of D3S1358, vWA, D165539, CSF1PO, TPDX, Amelogenin, D8S1179, D21S11, D18551, D2S441, D195433, THO1, FGA, D22S1045, D5S818, D135317, D7S820, SE33, D10S1248, D1S1656, D125391, D2S1338, Penta D and Penta; one or more Y-chromosome short tandem repeat loci selected from the group consisting of DYS576, DYS389I, DYS448, DYS389II, DYS19, DYS391, DYS481, DYS549, DYS533, DYS438, DYS437, DYS570, DYS635, DYS390, DYS439, DYS392, DYS643, DYS393, DYS458, DYS385a/b, DYS456 and Y-GATA-H4; and one or more X-chromosome short tandem repeat loci selected from the group consisting of DXS7132 (SEQ ID NO: 1), DXS7423 (SEQ ID NO: 2), DXS8378 (SEQ ID NO: 3), DXS10074 (SEQ ID NO: 4), DXS10079 (SEQ ID NO: 5), DXS10101 (SEQ ID NO: 6), DXS10103 (SEQ ID NO: 7), DXS10134 (SEQ ID NO: 8), DXS10135 (SEQ ID NO: 9), DXS10146 (SEQ ID NO: 10), DXS10148 (SEQ ID NO: 11), and HPRTB (SEQ ID NO: 12).

13. A set of primers which amplify D3S1358, vWA, D165539, CSF1PO, TPDX, Amelogenin, D8S1179, D21S11, D18551, D2S441, D195433, THO1, FGA, D22S1045, D5S818, D135317, D7S820, SE33, D10S1248, D1S1656, D125391, D2S1338, Penta D, Penta E, DXS7132 (SEQ ID NO: 1), DXS7423 (SEQ ID NO: 2), DXS8378 (SEQ ID NO: 3), DXS10074 (SEQ ID NO: 4), DXS10079 (SEQ ID NO: 5), DXS10101 (SEQ ID NO: 6), DXS10103 (SEQ ID NO: 7), DXS10134 (SEQ ID NO: 8), DXS10135 (SEQ ID NO: 9), DXS10146 (SEQ ID NO: 10), DXS10148 (SEQ ID NO: 11), and HPRTB (SEQ ID NO: 12), DYS576, DYS389I, DYS448, DYS389II, DYS19, DYS391, DYS481, DYS549, DYS533, DYS438, DYS437, DYS570, DYS635, DYS390, DYS439, DYS392, DYS643, DYS393, DYS458, DYS385a/b, DYS456 and Y-GATA-H4.

14. A kit for DNA fingerprinting or profiling comprising a plurality of primers which amplify one or more autosomal chromosome short tandem repeat loci selected from the group consisting D3S1358, vWA, D165539, CSF1PO, TPDX, Amelogenin, D8S1179, D21S11, D18551, D2S441, D195433, THO1, FGA, D22S1045, D5S818, D135317, D7S820, SE33, D1051248, D1S1656, D125391, D2S1338, Penta D and Penta E; one or more X-chromosome short tandem repeat loci selected from the group consisting of DXS7132 (SEQ ID NO: 1), DXS7423 (SEQ ID NO: 2), DXS8378 (SEQ ID NO: 3), DXS10074 (SEQ ID NO: 4), DXS10079 (SEQ ID NO: 5), DXS10101 (SEQ ID NO: 6), DXS10103 (SEQ ID NO: 7), DXS10134 (SEQ ID NO: 8), DXS10135 (SEQ ID NO: 9), DXS10146 (SEQ ID NO: 10), DXS10148 (SEQ ID NO: 11), and HPRTB (SEQ ID NO: 12); and one or more Y-chromosome loci selected from the group consisting of DYS576, DYS389I, DYS448, DYS389II, DYS19, DYS391, DYS481, DYS549, DYS533, DYS438, DYS437, DYS570, DYS635, DYS390, DYS439, DYS392, DYS643, DYS393, DYS458, DYS385a/b, DYS456 and Y-GATA-H4.

15. A kit for DNA fingerprinting or profiling comprising a plurality of primers which amplify the autosomal chromosome short tandem repeat loci: D3S1358, vWA, D165539, CSF1PO, TPDX, Amelogenin, D8S1179, D21S11, D18551, D2S441, D195433, THO1, FGA, D22S1045, D5S818, D135317, D7S820, SE33, D10S1248, D1S1656, D125391, D2S1338, Penta D and Penta E; X-chromosome short tandem repeat loci: DXS7132 (SEQ ID NO: 1), DXS7423 (SEQ ID NO: 2), DXS8378 (SEQ ID NO: 3), DXS10074 (SEQ ID NO: 4), DXS10079 (SEQ ID NO: 5), DXS10101 (SEQ ID NO: 6), DXS10103 (SEQ ID NO: 7), DXS10134 (SEQ ID NO: 8), DXS10135 (SEQ ID NO: 9), DXS10146 (SEQ ID NO: 10), DXS10148 (SEQ ID NO: 11), and HPRTB (SEQ ID NO: 12); and Y-chromosome short tandem repeat loci: DYS19, DYS385 a/b, DYS387S1 a/b, DYS389I, DYS389II, DYS390, DYS391, DYS392, DYS393, DYS437, DYS438, DYS439, DYS448, DYS449, DYS456, DYS458, DYS460, DYS481, DYS518, DYS533, DYS570, DYS576, DYS627, DYS635, DYS549, DYS643 and Y-GATA-H4.

16. The kit of claim 14, wherein plurality of primers are contained together in a single formulation.

17. The kit of claim 15, wherein each of the primers among the plurality of primers are contained in a single formulation.

18. A kit for DNA fingerprinting or profiling comprising: a plurality of primers which amplify one or more X-chromosome short tandem repeat loci; a plurality of primers which amplify one or more Y-chromosome short tandem repeat loci; a plurality of primers which amplify one or more autosomal chromosome short tandem repeat loci; at least one DNA polymerase; at least one DNA ladder, at least one DNA control; and at least one allelic ladder.

19. The kit according to claim 18, wherein the plurality of primers which amplify one or more Y-chromosome short tandem repeat loci comprises DXS7132 (SEQ ID NO: 1), DXS7423 (SEQ ID NO: 2), DXS8378 (SEQ ID NO: 3), DXS10074 (SEQ ID NO: 4), DXS10079 (SEQ ID NO: 5), DXS10101 (SEQ ID NO: 6), DXS10103 (SEQ ID NO: 7), DXS10134 (SEQ ID NO: 8), DXS10135 (SEQ ID NO: 9), DXS10146 (SEQ ID NO: 10), DXS10148 (SEQ ID NO: 11), and HPRTB (SEQ ID NO: 12), wherein the plurality of primers which amplify one or more X-chromosome short tandem repeat loci comprises DYS576, DYS389I, DYS448, DYS389II, DYS19, DYS391, DYS481, DYS549, DYS533, DYS438, DYS437, DYS570, DYS635, DYS390, DYS439, DYS392, DYS643, DYS393, DYS458, DYS385a/b, DYS456 and Y-GATA-H4, and wherein the plurality of primers which amplify one or more autosomal chromosome short tandem repeat loci comprises D3S1358, vWA, D165539, CSF1PO, TPDX, Amelogenin, D8S1179, D21S11, D18551, D2S441, D195433, THO1, FGA, D22S1045, D5S818, D135317, D7S820, SE33, D10S1248, D1S1656, D125391, D2S1338, Penta D and Penta E.

20. The kit according to claim 18, wherein the at least one DNA polymerase comprises a taq polymerase, wherein the at least one DNA ladder comprises a 600 bp ladder, and wherein the at least one DNA control comprises human female control DNA 9947A.

21. The kit according to claim 18, wherein said one or more allelic ladders comprise allelic ladder X short tandem repeats, allelic ladder Y short tandem repeats, and allelic ladder autosomal short tandem repeats.

22. The kit according to claim 19, wherein said one or more allelic ladders comprise allelic ladder X short tandem repeats, allelic ladder Y short tandem repeats, and allelic ladder autosomal short tandem repeats.

23. The kit according to claim 20, wherein said one or more allelic ladders comprise allelic ladder X short tandem repeats, allelic ladder Y short tandem repeats, and allelic ladder autosomal short tandem repeats.


SEQUENCE LISTING (12 sequences)

SEQ ID NO: 1 - DNA, 52 bp, Artificial Sequence
  Synthetic
tctatctatctatctatctatctatctatctatctatctatctatctatcta

SEQ ID NO: 2 - DNA, 68 bp, Artificial Sequence
  Synthetic
tccatccatccatctgtccttccatccatccatccatccatccatccatccatccatccatccatcca

SEQ ID NO: 3 - DNA, 48 bp, Artificial Sequence
  Synthetic
ctatctatctatctatctatctatctatctatctatctatctatctat

SEQ ID NO: 4 - DNA, 56 bp, Artificial Sequence
  Synthetic
aagaaagaaagaaagaaagaaagaaagaaagaaagaaagaaagaaagaaagaaaga

SEQ ID NO: 5 - DNA, 104 bp, Artificial Sequence
  Synthetic
agagagagagagtgaaagagagaaagaaagaaagaaagaaagaaagaaagaaagaaagaaagaaagaaagaaagaaagaaagaaagaaagagagaaagaaagaa

SEQ ID NO: 6 - DNA, 135 bp, Artificial Sequence
  Synthetic
aaagaaagaaaggaaagaaggaaagaaagaaaagaaagaaagaaagaaaaagaaaagaaagaaagaaagaaagaaaaagaaaaagaaagaaagaaagaaagaaagaaagaaagaaagaaagaaagaaagaaagaa

SEQ ID NO: 7 - DNA, 80 bp, Artificial Sequence
  Synthetic
tagatagactgacagatagatagatagatagatagatagatagatagatagatagatagacagacagacagacagataga

SEQ ID NO: 8 - DNA, 182 bp, Artificial Sequence
  Synthetic
gaaagaaagaaagagagaaagaaagaaagaaaaagaaagagagaaagaaagaaagaaagagagacagagacagagacagagaaagtaagaaagaaagaaaaaagaaagaaagaaagaaaaaagaaagaaagaaagaaagaaagaaagaaagaaagaaagaaagaaagaaagaaagaaagaaa

SEQ ID NO: 9 - DNA, 97 bp, Artificial Sequence
  Synthetic
aagaaagaaagagaaaggaaagaaagaaagaaagaaagaaagaaagaaagaaagaaagaaagaaagaaagaaagaaagaaagaaagaaagaaagaaa

SEQ ID NO: 10 - DNA, 131 bp, Artificial Sequence
  Synthetic
ttccttccttcctttccttccttcctttcctcccttccttcctcccttcttctttcttccttcctttcttctttctttcttcctttctttctttctttctttctttctttctttctttcttttctttcttt

SEQ ID NO: 11 - DNA, 96 bp, Artificial Sequence
  Synthetic
ggaaggaaggaaggaaaagaaagaaagaaagaaagaaagaaagaaagaaagaaagaaagaaagaaaagaaagaaagaaagnnnnnnnnaaggaagg

SEQ ID NO: 12 - DNA, 48 bp, Artificial Sequence
  Synthetic
agatagatagatagatagatagatagatagatagatagatagatagat